(19) United States
(12) Patent Application Publication
(10) Pub. No.: US 2021/0065912 A1
(43) Pub. Date: Mar. 4, 2021

(54) SYSTEM AND METHOD FOR FACILITATING DATA ANALYSIS PERFORMANCE

(71) Applicant: KONINKLIJKE PHILIPS N.V., EINDHOVEN (NL)

(72) Inventors: JAN JOHANNES GERARDUS DE VRIES, LEENDE (NL); JOEP JOSEPH BENJAMIN NATHAN VAN BERKEL, GRONSVELD (NL)

(21) Appl. No.: 16/644,630

(22) PCT Filed: Aug. 30, 2018
(86) PCT No.: PCT/EP2018/073285

§ 371 Date: Mar. 5, 2020

Related U.S. Application Data
(60) Provisional application No. 62/556,558, filed on Sep. 11, 2017.

(51) Int. Cl. G16H 50/70; G16H 10/60; G06F 16/22; G16H 50/30; G06F 16/901; G06Q 30/02

(52) U.S. Cl. CPC G16H 50/70; G16H 10/60; G06Q 30/0206; G16H 50/30; G06F 16/9024; G06F 16/2264

(57) ABSTRACT

Provided is a system and method for facilitating data analysis performance with respect to analysis of individuals having one or more health conditions. The system comprises one or more processors configured to obtain profile information regarding profiles, each of the profiles indicating one or more health conditions or an individual having one or more health conditions; obtain probability information regarding probabilities of an individual developing health conditions, each of the probabilities being a probability of an individual developing a health condition; for each profile of the profiles, determine a relationship between the profile and one or more other profiles that are different from the profile with respect to at least one health condition, the determination of the relationship being based on one of the probabilities of an individual developing the at least one health condition; and generate a data structure representative of the profiles based on the determined relationships.

### BACKGROUND

### 1. Field

The present disclosure pertains to a system and method for facilitating data analysis performance, including improvements to clustering performance or other data analysis performance.

### 2. Description of the Related Art

Clustering technologies are often employed to identify “clusters” or groups/sub-groups with respect to a data collection. For example, clustering may involve grouping a set of objects in such a way that objects in the same group are more similar (in some aspect) to one other, as compared to those in other groups. Clustering is generally used for data mining and frequently used for statistical data analysis in many technological areas, including machine learning, pattern recognition, bioinformatics, medical technologies, or other technological areas. In general, to perform well, clustering technologies rely on reliable measures of similarity or dissimilarity or the assignment of values of such measures to objects. Typical measures used for clustering technologies, however, fail to produce reliable results in a number of scenarios, such as various use cases involving patient or disease data analysis. These and/or other drawbacks exist.

### SUMMARY

Accordingly, one or more aspects of the present disclosure relate to a system for facilitating clustering performance with respect to analysis of individuals having one or more health conditions. The system includes one or more hardware processors configured by machine-readable instructions to: obtain profile information regarding profiles, each of the profiles indicating one or more health conditions or an individual having one or more health conditions; obtain probability information regarding probabilities of an individual developing health conditions, each of the probabilities being a probability of an individual developing a health condition; for each profile of the profiles, determine a relationship between the profile and one or more other profiles that are different from the profile with respect to at least one health condition, the determination of the relationship being based on at least one of the probabilities of an individual developing the at least one health condition; and generate a data structure representative of the profiles based on the determined relationships.

Another aspect of the present disclosure relates to a method for facilitating data analysis performance with respect to analysis of individuals having one or more health conditions with a system. The system includes one or more hardware processors configured by machine-readable instructions, the method including: obtaining profile information regarding profiles, each of the profiles indicating one or more health conditions or an individual having one or more health conditions; obtaining probability information regarding probabilities of an individual developing health conditions, each of the probabilities being a probability of an individual developing a health condition; for each profile of the profiles, determining a relationship between the profile and one or more other profiles that are different from the profile with respect to at least one health condition, the determination of the relationship being based on at least one of the probabilities of an individual developing the at least one health condition; and generating a data structure representative of the profiles based on the determined relationships.

Still another aspect of the present disclosure relates to a system for facilitating data analysis performance with respect to analysis of individuals having one or more health conditions. The system includes: means for obtaining profile information regarding profiles, each of the profiles indicating one or more health conditions or an individual having one or more health conditions; means for obtaining probability information regarding probabilities of an individual developing health conditions, each of the probabilities being a probability of an individual developing a health condition; means for determining, for each profile of the profiles, a relationship between the profile and one or more other profiles that are different from the profile with respect to at least one health condition, the determination of the relationship being based on at least one of the probabilities of an individual developing the at least one health condition; and means for generating a data structure representative of the profiles based on the determined relationships.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 illustrates a system to facilitate data analysis performance, in accordance with one or more embodiments.

FIG. 2 illustrates an example of a representation of patient information, in accordance with one or more embodiments.

FIG. 3 is a schematic illustration of an example of a scaled Cityblock distance, in accordance with one or more embodiments.

FIG. 4 is a schematic illustration of an example of patient clustering, in accordance with one or more embodiments.

FIG. 5 is a schematic illustration of patient clusters based on disease profiles, in accordance with one or more embodiments.

FIG. 6 illustrates a method for facilitating data clustering performance, in accordance with one or more embodiments.

### DETAILED DESCRIPTION OF EXEMPLARY EMBODIMENTS

As used herein, the singular form of “a,” “an,” and “the” include plural references unless the context clearly dictates otherwise. As used herein, the term “or” means “and/or” unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

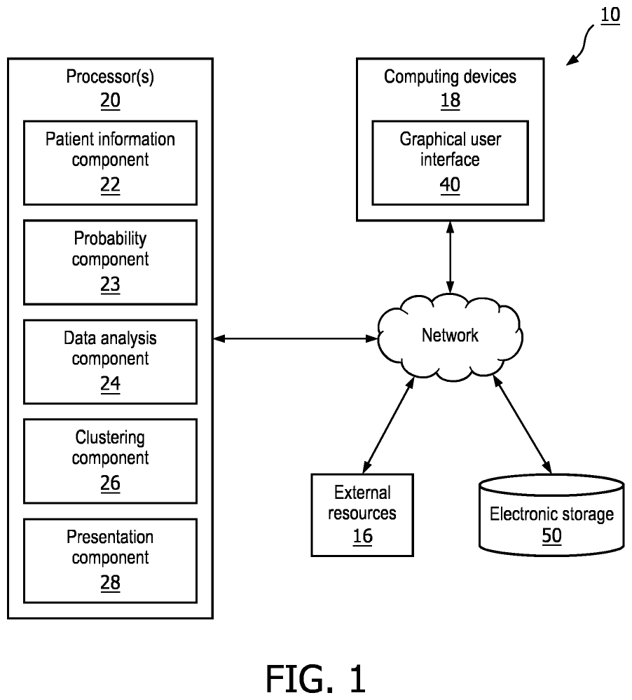

FIG. 1 is a schematic illustration of a system 10 configured to facilitate data analysis performance. In some embodiments, system 10 provides data analysis of data specific to patients. Generally, healthcare providers (e.g., hospitals) are in a continuous effort to optimize their care, lower cost, improve patient experience, and search for new subgroups of patients (from the overall patient population) to serve. Generally, data analysis is used to identify such subgroups of patients. For example, clustering techniques are used to identify such subgroups, but their output highly depends on a good choice of a dissimilarity measure (i.e., a mathematical representation that describes how dissimilar two patients are). Such dissimilarity measures have been developed over time, but many are not applicable to the description of patients in terms of the binary representation of their multi-morbidity status.

In some embodiments, system 10 provides an approach that is tailored to reflect differences in disease profiles (or other profiles) of patients. In some embodiments, system 10 provides sets of similar patients (in terms of their disease profiles) yet different from the vast majority that has common disease profiles. In some embodiments, system 10 allows identification of groups/subgroups of data that provides information clinically relevant to the end-user. In some embodiments, system 10 is configured to provide clustering techniques that are expected to enhance the ability to identify groups/subgroups of patients that represent patients with similar disease profiles, yet different from the majority of patients that show common disease profiles. In some embodiments, system 10 is configured to model the probabilities of developing each disease of interest (or other health condition of interest) and taking this into account when describing dissimilarity of patients. In some embodiments, severity, and/or costs related to each disease of interest are also taken in consideration when describing dissimilarity of patients. Identifying such patient groups can help healthcare providers tailor their healthcare offering better to their patient population. It should be noted that, although some embodiments are described herein with respect to improving clustering performance (e.g., accuracy, reliability, etc., of clustering results), the operations and features described herein may be applied in other embodiments to facilitate performance of other data analysis aspects.

In some embodiments, system 10 may generate a data structure on which performance of clustering or other processing on a data collection may be based. The generated data structure may include a graph-based data structure (e.g., a graph), a vector-based data structure (e.g., a list or set of vectors, etc.), or other data structure. The generated data structure may represent profiles indicating one or more health conditions (e.g., diseases or other health conditions), profiles indicating individuals having one or more health conditions, or other profiles. In some embodiments, probability information may be used to create or modify the data structure to tailor the data structure and subsequent clustering or processing based on the data structure. The data structure may, for example, provide one or more clustering algorithms with probability-related measures of similarity or dissimilarity to enable such clustering algorithms to produce more relevant or more accurate results.

In some embodiments, the probability information may indicate a first probability of an individual developing a first health condition, a second probability of an individual developing a second health condition, and so on. In some embodiments, system 10 may utilize the probabilities to determine, for each profile of a set of profiles, a relationship between the profile and one or more other profiles. As an example, system 10 may determine a distance (e.g., a dissimilarity distance, a similarity distance, etc.) between the profile and one or more other profiles that are different from the profile with respect to at least one health condition (e.g., with respect to only one health condition, with respect to more than one health condition, etc.) based on respective probabilities of an individual developing the differing health condition(s). In some embodiments, the distance may be determined based on severity related to the at least one health condition, and/or one or more costs related to the at least health condition. In one use case, where the data structure includes edges connecting one or more nodes or data points corresponding to the profiles, system 100 may assign the determined distances to the edges respectively linking the profile and the other profiles in the data structure. In this way, if the data structure is used to perform clustering on a data collection of individuals having one or more health conditions to identity groups/subgroups of individuals, system 10 may use the assigned distances to produce the resulting groups/subgroups so that those results more accurately reflect a health-condition-related similarity of individuals within the same group or dissimilarity between individuals of different groups.

In some embodiments, system 10 includes external resources 16, computing devices 18, processors 20, electronic storage 50, and/or other components. External resources 16 include sources of patient and/or other information. In some embodiments, external resources 16 include sources of patient and/or other information, such as databases, websites, etc., external entities participating with system 10 (e.g., a medical records system of a healthcare provider that stores medical history information for populations of patients), one or more servers outside of system 10, a network (e.g., the internet), electronic storage, equipment related to Wi-Fi technology, equipment related to Bluetooth® technology, data entry devices, sensors, scanners, and/or other resources. For example, in some embodiments, external resources 16 may include a database where medical history information for a plurality of patients are stored, and/or other sources of information such as sources of information related to patient demographics, diagnoses, problem lists, treatments, lab data, and/or other information. In some embodiments, the patient information includes initial vital signs of patients, treatments provided to the patients with the respective initial vital signs, respective vital signs resulting from the treatments, and/or other information. In some implementations, some or all of the functionality attributed herein to external resources 16 may be provided by resources included in system 10. External resources 16 may be configured to communicate with processor 20, computing devices 18, electronic storage 50, and/or other components of system 10 via wired and/or wireless connections, via a network (e.g., a local area network and/or the internet), via cellular technology, via Wi-Fi technology, and/or via other resources.

Computing devices 18 are configured to provide interfaces between caregivers (e.g., doctors, nurses, friends, family members, etc.), patients, and/or other users, and system 10. In some embodiments, individual computing devices 18 are, and/or are included, in desktop computers, laptop computers, tablet computers, smartphones, and/or other computing devices associated with individual caregivers, patients, and/or other users. In some embodiments, individual computing devices 18 are, and/or are included, in equipment used in hospitals, doctor's offices, and/or other medical facilities to patients; test equipment; equipment for treating patients; data entry equipment; and/or other devices. Computing devices 18 are configured to provide information to, and/or receive information from, the caregivers, patients, and/or other users. For example, computing devices 18 are configured to present a graphical user interface 40 to the caregivers to facilitate display representations of the data analysis, and/or other information. In some embodiments, graphical user interface 40 includes a plurality of separate interfaces associated with computing devices 18, processor 20 and/or other components of system 10; multiple views and/or fields configured to convey information to and/or receive information from caregivers, patients, and/or other users; and/or other interfaces.

In some embodiments, computing devices 18 are configured to provide graphical user interface 40, processing capabilities, databases, and/or electronic storage to system 10. As such, computing devices 18 may include processors 20, electronic storage 50, external resources 16, and/or other components of system 10. In some embodiments, computing devices 18 are connected to a network (e.g., the internet). In some embodiments, computing devices 18 do not include processors 20, electronic storage 50, external resources 16, and/or other components of system 10, but instead communicate with these components via the network. The connection to the network may be wireless or wired. For example, processor 20 may be located in a remote server and may wirelessly cause display of graphical user interface 40 to the caregivers on computing devices 18. As described above, in some embodiments, an individual computing device 18 is a laptop, a personal computer, a smartphone, a tablet computer, and/or other computing devices. Examples of interface devices suitable for inclusion in an individual computing device 18 include a touch screen, a keypad, touch-sensitive and/or physical buttons, switches, a keyboard, knobs, levers, a display, speakers, a microphone, an indicator light, an audible alarm, a printer, and/or other interface devices. The present disclosure also contemplates that an individual computing device 18 includes a removable storage interface. In this example, information may be loaded into a computing device 18 from removable storage (e.g., a smart card, a flash drive, a removable disk, etc.) that enables the caregivers, patients, and/or other users to customize the implementation of computing devices 18. Other exemplary input devices and techniques adapted for use with computing devices 18 include, but are not limited to, an RS-232 port, an RF link, an IR link, a modem (telephone, cable, etc.), and/or other devices.

Processor 20 is configured to provide information processing capabilities in system 10. As such, processor 20 may include one or more of a digital processor, an analog processor, a digital circuit designed to process information, an analog circuit designed to process information, a state machine, and/or other mechanisms for electronically processing information. Although processor 20 is shown in FIG. 1 as a single entity, this is for illustrative purposes only. In some embodiments, processor 20 may include a plurality of processing units. These processing units may be physically located within the same device (e.g., a server), or processor 20 may represent processing functionality of a plurality of devices operating in coordination (e.g., one or more servers, one or more computing devices 18 associated with caregivers, a piece of hospital equipment, devices that are part of external resources 16, electronic storage 50, and/or other devices.)

In some embodiments, processor 20, external resources 16, computing devices 18, electronic storage 50, and/or other components may be operatively linked via one or more electronic communication links. For example, such electronic communication links may be established, at least in part, via a network such as the Internet, and/or other networks. It will be appreciated that this is not intended to be limiting, and that the scope of this disclosure includes embodiments in which these components may be operatively linked via some other communication media. In some embodiments, processor 20 is configured to communicate with external resources 16, computing devices 18, electronic storage 50, and/or other components according to a client/server architecture, a peer-to-peer architecture, and/or other architectures.

As shown in FIG. 1, processor 20 is configured via machine-readable instructions to execute one or more computer program components. The computer program components may include one or more of a patient information component 22, a probability component 23, a data analysis component 24, a clustering component 26, a presentation component 28, and/or other components. Processor 20 may be configured to execute components 22, 23, 24, 26, and/or 28 by software; hardware; firmware; some combination of software, hardware, and/or firmware; and/or other mechanisms for configuring processing capabilities on processor 20.

It should be appreciated that although components 22, 23, 24, 26, and 28 are illustrated in FIG. 1 as being co-located within a single processing unit, in embodiments in which processor 20 includes multiple processing units, one or more of components 22, 23, 24, 26, and/or 28 may be located remotely from the other components. The description of the functionality provided by the different components 22, 23, 24, 26, and/or 28 described below is for illustrative purposes, and is not intended to be limiting, as any of components 22, 23, 24, 26, and/or 28 may provide more or less functionality than is described. For example, one or more of components 22, 23, 24, 26, and/or 28 may be eliminated, and some or all of its functionality may be provided by other components 22, 23, 24, 26, and/or 28. As another example, processor 20 may be configured to execute one or more additional components that may perform some or all of the functionality attributed below to one of components 22, 23, 24, 26, and/or 28.

In some embodiments, patient information component 22 is configured to obtain patient information related to a plurality of patients. In some embodiments, patient information may include demographic information (e.g., gender, ethnicity, age, etc.), vital signs information (e.g., heart rate, temperature, respiration rate, etc.), medical/health condition information (e.g., a disease type, severity of the disease, stage of the disease, categorization of the disease, symptoms, behaviors, readmission, relapse, death, etc.), treatment information (e.g., length of treatment, length of stay in a medical facility, medications, interventions, costs of treatment, etc.), outcome information (e.g., discharge date, prognosis, readmission date, etc.), and/or other information. It should be noted that the patient information described above is not intended to be limiting. A large number of information related to patients may exist and may be used with system 10 in accordance with some embodiments. For example, users may choose to customize system 10 and include any type of patient data they deem relevant.

In some embodiments, patient information component 22 may be configured to obtain/extract information from one or more databases. In some embodiments, different databases may contain different information about one patient or about multiple patients. In some embodiments, some databases may be associated with specific patient information (e.g., a medical condition, a demographic characteristic, a treatment, an outcome, a vital sign information, etc.) or associated with a set of patient information (e.g., a set of medical conditions, a set of demographic characteristics, etc.). In some embodiments, patient information component 22 may be configured to obtain/extract the patient information from external resources 16 (e.g., one or more external databases included in external resources 16), electronic storage 50 included in system 10, one or more medical devices (not shown), and/or other sources of information.

In some embodiments, patient information component 22 may be configured to process the patient information into a desired format. For example, in some embodiments, patient information (for all the patient population) may be modified to have a similar consistent format (even if the patient information is obtained from different databases). In some embodiments, patient information component 22 may be configured to normalize the patient information. In some embodiments, patient information component 22 may be configured to organize patient information into profiles, such as patient profiles, health condition profiles (e.g., disease profiles), or other profiles. In some embodiments, patient information component 22 may be configured to obtain profile information regarding profiles (e.g., from one or more data bases). In some embodiments, profile information may include information regarding 500 or more profiles, 1000 or more profiles, 10000 or more profiles, 100000 or more profiles, 1000000 or more profiles, or other number of profiles. In some embodiments, each one of the profiles indicates one or more health conditions. In some embodiments, each profile indicates an individual having one or more health conditions. In some embodiments, for example, each profile is associated to an individual, and the profile indicates which health conditions the patient has and/or which he does not have.

In some embodiments, patient information may be represented by assigning a vector to each patient (and/or to each profile) in the patient population. In some embodiments, each patient vector includes one or more dimensions. In some embodiments, each of the dimensions indicates whether a patient has one or more health conditions (e.g., a predetermined set of medical conditions).

For example, a patient may be described in association with a set of chosen (e.g., chronic) diseases with a vector that represents the presence of a disease (from the chosen diseases), in the patient, with a “1” and the absence of the disease in the patient with a “0”. As a result, the patient may be represented by a point in a multi-dimensional binary space. In other words, the patient is associated with a multi-dimensional vector, where the number of the dimensions is the number of chosen diseases, and where each dimension indicates whether the patient has or does not have a given disease. For example, with respect to a three-dimensional space, each dimension may correspond to a disease of interest. A patient represented by vector (0,1,1) indicates that the patient does not suffer from the first disease, and suffers from the second and third diseases. In other examples, a vector having N-dimensions may indicate whether the patient has one or more of N-diseases. In one use case, vector (0,0,0,0,0) represents a profile where a patient does not have any of five particular diseases to which the five dimensions correspond. In another use case, vector (1,1,1,1,1,1,1) represents a profile where a patient has all seven particular diseases to which the seven dimensions correspond.

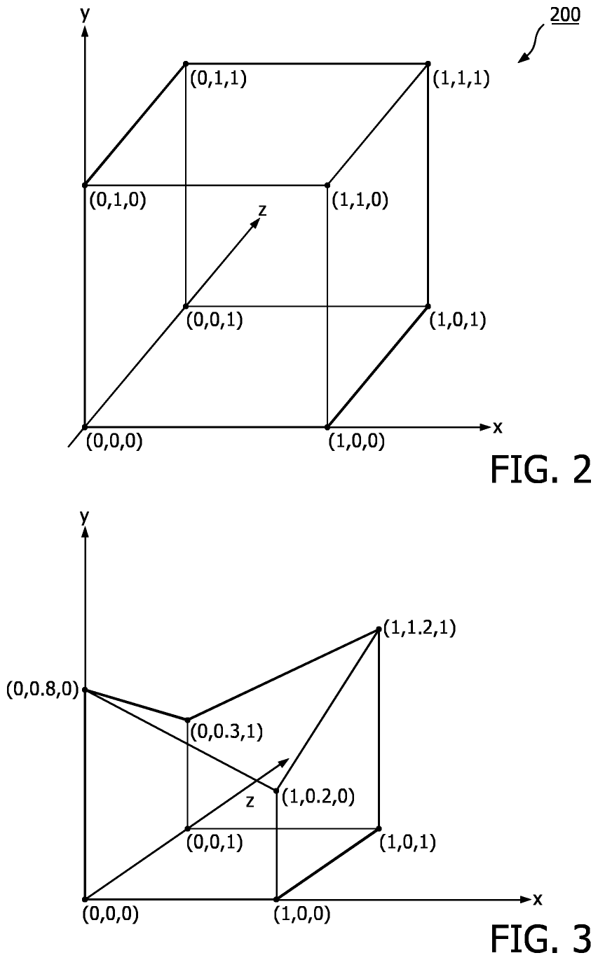

FIG. 2 illustrates an example of a representation 200 of patient profiles (or disease profiles), in accordance with one or more embodiments. In this example, axis x represents a first disease of interest, axis y represents a second disease of interest, and axis z represents a third disease of interest. For example, vector (0,1,1) represents a patient with absence of the first disease and presence of the other two diseases. A second patient represented by vector (1,1,0) has the first and the second disease and does not have the third disease. These two patients share the second disease; however, they are different in terms of the first and third disease. In some embodiments, for each profile of the profiles, a relationship between the profile and one or more other profiles that are different from the profile may be determined (e.g., with respect to at least one health condition or disease.). In some embodiments, determining relationships between profiles includes determining distances between the profiles. In some embodiments, the distances may be assigned to the respective profiles. For example, in some embodiments, Cityblock distance may be used to measure the patients' dissimilarity. Cityblock distance counts the number of differing elements in the binary descriptors, or mathematically: d_{cityblock}(P,Q)=Σ|Pᵢ−Qᵢ| for P,Q∈{0,1}^{N} where N is the number of diseases under consideration (N=3 in the example of FIG. 2). The Cityblock distance in this example may be characterized as a walk along the blue edges of cube 200. In the context of multi-morbidity, going from one disease profile to another, diseases present in the first but not in the second disease profile are eliminated, and diseases that are present in the second but not the first are gained. Naturally, a vector may have any number of dimensions (N) where the points that patients can be represented with are located on the vertices of an N-dimensional hypercube.

Returning to FIG. 1, in some embodiments, probability component 23 is configured to obtain probability information regarding probabilities of an individual (e.g., a patient) developing health conditions. In some embodiments, each of the probabilities is a probability of an individual developing a health condition. In some embodiments, probability component 23 may be configured to obtain/calculate a probability of the patient developing a given medical condition, responsive to the patient not having the given medical condition (e.g., how easy it is to develop the disease). In some embodiments, probability component 23 may be configured to obtain a probability of the patient getting better from given medical condition (e.g., how easy it is to lose the disease.) In some embodiments, an example for measuring probability of the patient developing a given medical condition may be achieved by analyzing the descriptive statistics of the patient population and derive the prevalence per disease (pₓ=P(X=1)). Low probabilities may indicate that it is “difficult to develop the disease,” and therefore not a lot of people have the disease. High probabilities may indicate that it is “easy to develop the disease,” and therefore a lot of people may have the disease.

In some embodiments, probability component 23 may be configured to obtain the probability information by determining a first probability of an individual having a first set of health conditions developing a second health condition not included in the first set of health conditions. For example, in some embodiments, probability component 23 may be configured to obtain a “conditional” probability of the patient developing a given medical condition based on one or more medical conditions that the patient already has (the probability is conditional to the patient having one or more medical conditions). For example, in some embodiments, for some disease profiles it might be easier to develop a particular disease (comorbidity) than for other disease profiles. That is, if a patient has a set of diseases, he may be more likely to gain another disease compared to the case where he does not have this set of diseases.

In some embodiments, data analysis component 24 may be configured to determine, for each profile of the profiles, a relationship between the profile and one or more other profiles that are different from the profile with respect to at least one health condition. In some embodiments, the determination of the relationship between the profiles is based on at least one of the probabilities of an individual (associated with the profile) developing the at least one health condition. In some embodiments, the determination of the relationship is based (instead or in addition to the at least one of the probabilities) on severity and/or costs related to the at least one health condition. In some embodiments, determining a relationship for each profile of the profiles includes determining distances between the profiles. In some embodiments, the determination of the distances is based on severity and/or costs related to the at least one health condition. In some embodiments, the distances may be assigned to the respective profiles.

In some embodiments, the probability information includes a probability of an individual having a first set of health conditions developing a second health condition not included in the first set of health conditions (conditional probability). Data analysis component 24 may be configured to determine a relationship (e.g., a distance or other relationship) between a first profile and a second profile, where the first profile corresponds to the first set of health conditions that includes a first health condition, and where the second profile corresponds to a second set of health conditions that includes the first health condition and the second health condition. As an example, the second set of health conditions may include the second health condition and all health conditions in the first set of health conditions. In one use case, for instance, the first profile may correspond to vector (0,0,0,1), and the second profile may correspond to vector (0,0,1,1). In another use case, the first profile may correspond to vector (0,0,1,1), and the second profile may correspond to vector (0,1,1,1). In some embodiments, data analysis component 24 may determine a relationship between the first profile and a third profile, a relationship between the first profile and a fourth profile, and so on. The relationship between the first profile and the third profile may be determined based on a probability of an individual having the first set of health conditions developing a third health condition of a third set of health conditions to which the third profile corresponds. The relationship between the first profile and the fourth profile may be determined based on a probability of an individual having the first set of health conditions developing a fourth health condition of a fourth set of health conditions to which the fourth profile corresponds.

In some embodiments, data analysis component 24 may be configured to generate a data structure representative of the profiles based on the determined relationships. In some embodiments, the generated data structure may include a graph-based data structure, a vector-based data structure, or other data structure. In some embodiments, the data structure includes edges that reflect the assigned distances. For example, where each patient (profile) is assigned a vector that includes one or more dimensions indicating whether a patient has one or more medical conditions, data analysis component 24 may be configured to weigh a dimension of the medical condition in the patient vector with the probability of the patient developing the given medical condition to create a modified patient vector. In some embodiments, data analysis component 24 may be configured to weigh the dimension of the medical condition in the patient vector (instead or in addition to the probability) with severity and/or costs related to the medical condition to create a modified patient vector. For example, in some case where a patient already has a medical condition, the dimension between the patient profile and another patient profile may be weighed based on the severity of the medical condition in the patient vs the severity of the medical condition in the other patient. For example, two patient may have the same disease but at different stages of the disease. An advanced stage of the disease may have more weight than an early stage of the disease, for example. The same principle can be applied to the costs of the medical condition (i.e. the distance between two profiles can be weighed based on the costs related to medical condition for the two profiles). The same disease may have different costs related to the disease for different patients. For example, a patient who is admitted in a hospital may have different costs related to the disease than a patient is at home and only visits the hospital for treatment. Other factors that may affect the cost of treatments may include proximity to care providers, access to medication, access to technology, geographic areas, and/or other factors.

In these embodiments, to obtain a distance between two patients (two vectors) Cityblock distance “walking the edges” may be used in similar way as d(P,Q)=Σ|Pᵢ−Qᵢ|, however now in the scaled space (i.e., P,Q∈[0,1]^{N}). For example, in the case of the example of FIG. 2, the cube 200 may be scaled linearly using the probability of developing the disease (1−p) such that all the vertices of certain planes of the cube would be moved (or stretched). In the example of FIG. 2, data analysis component 24 may be configured to weigh the edges of the cube with a value that represents the probability of developing the disease of which the axis is parallel to the edge (by integrating “how easy is it to develop a disease” parameter). For example, all the horizontally depicted edges describe (along axis x from left to right) the development of condition x, the vertical edges describe the development of condition y, and the diagonal edges describe the development of condition z. The cube 200 will be stretched in each direction x to size (1−pₓ). As a result of this scaling, diseases that are very common will naturally group together while less common diseases will be moved away. Therefore, clustering approaches will find a big cluster of common diseases but also satellite clusters that represent the patients with combinations of less common diseases. Experiments show that satellite clusters of large size may still be found (which is one indicator of clinical relevance.)

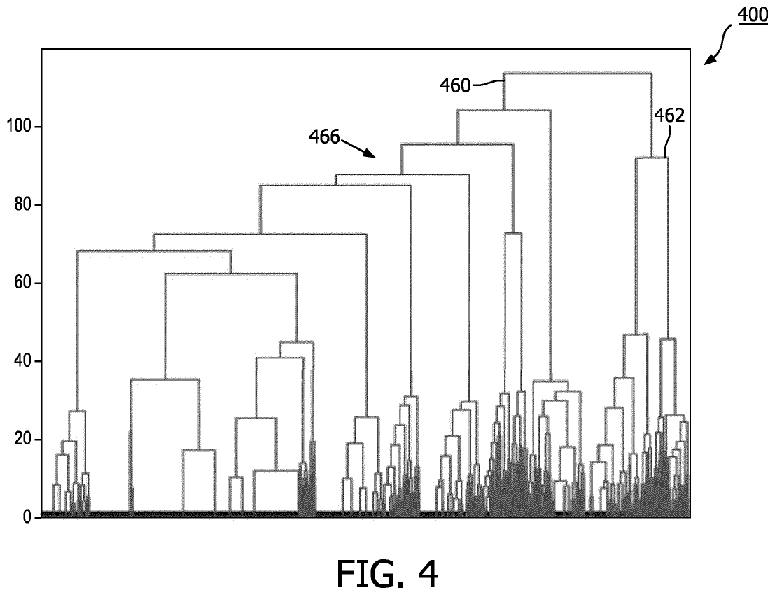

In some embodiments, data analysis component 24 may be configured to weigh a dimension of the medical condition in the patient vector with the “conditional” probability of the patient developing the medical condition to create a modified patient vector. Conditional probabilities pₓ=P(X=1|Y, Z, . . . ) may use a different way of calculating the distance measure as the cube (of FIG. 2) is now, generally, not scaled in a symmetrical way anymore and thus finding the distance between two vertices requires finding the shortest path in the graph spanned by the scaled edges. This can be done by using a minimum-path-finding algorithm such as Dijkstra's algorithm. d (P, Q) is defined as the length of the shortest path between P and Q. A representation of the cube (of FIG. 2) is scaled (or stretched) non-linearly by moving the vertices individually using the probability of developing the disease (1−p). FIG. 3 illustrates an example of a scaled Cityblock distance using conditional probabilities. FIG. 3 is a vector-based data structure where the edges reflect the assigned distances (based on the probabilities calculations). In some embodiments, data analysis component 24 may be configured to further weigh the dimension of the medical condition in the patient vector (instead or in addition to the “conditional” probability) with severity and/or costs related to the medical condition to create a modified patient vector. As can be seen for FIG. 3, all the points have more degrees of freedom and can move individually (as opposed to scaling the distance using probabilities where all the vertices of certain planes of the cube would have moved). In the example of FIG. 3, patient vector (0,1,1) became (0,0.3,1) and patient vector (1,1,0) became (1,0.2,0).

In some embodiments, clustering component 26 is configured to perform clustering of a data collection representative of individuals to obtain one or more groups of individuals. In some embodiments, clustering is based on the generated data structure. For example, clustering component 26 may be configured to cluster one or more patients (or profiles) based on a distance between the patients (or distance between the patients vectors as described above). In some embodiments, the patients in the patient population are organized into pairs representing a cluster based on the distance between patients. For example, two patients may form a pair if the distance between them reaches a predetermined distance threshold value (e.g., this value may be determined by a user based on the types of the medical diseases in the set of medical conditions or based on the patients in the patient population, or based on other factors). In some embodiments, a distance between two pairs of patients (two clusters) is obtained. The pair of patients may be grouped in a cluster based on the obtained distance (e.g., based on the distance threshold value, or a different distance threshold value). In some embodiments, this process of clustering patients is continued until all the patients are clustered.

In some embodiments, presentation component 28 is configured to cause a presentation related to data analysis performed by system 10. In some embodiments, the presentation is caused to be provided on graphical user interface 40 and/or other user interfaces. In some embodiments, for example, the presentation includes graphical or other representations of the patient information (e.g., normalized in a vector format representing the patient with a disease profile as shown in FIG. 2 and FIG. 3). In some embodiments, presentation component 28 may be configured to cause presentation of the scaled Cityblock dimensions (e.g., scaled based on obtained probabilities or obtained conditional probabilities). In some embodiments, presentation component 28 may be configured to cause presentation of patient clustering.

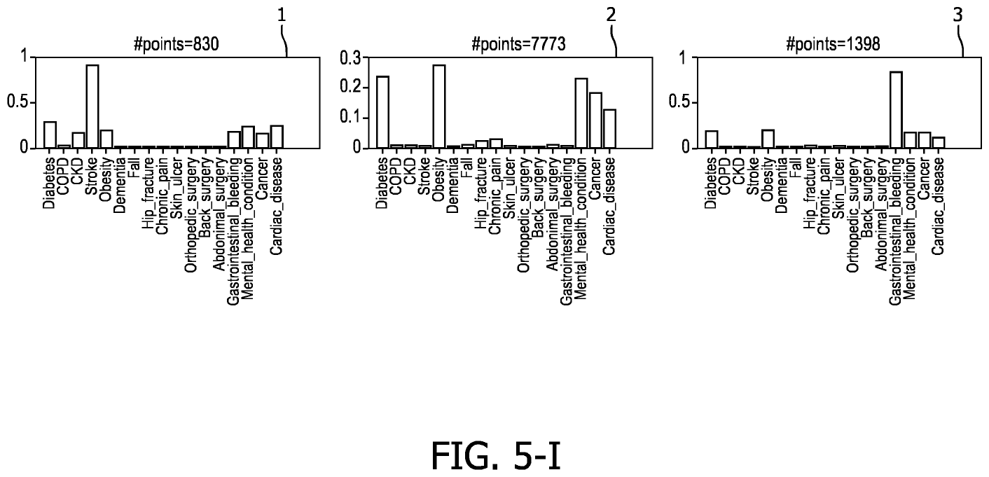

FIG. 4 illustrates an example of a graph 400 of patient clustering. The graph of FIG. 4 is a Dendrogram. A distance-based clustering with agglomerative hierarchical clustering (similar to the one described above) was used in this example. The distances were obtained using the scaled Cityblock distance method described above. An analysis of disease profiles covering 17 chronic diseases of over 14,000 patients was performed in this example (each of the patients were identified by means of a 17-dimensional binary factor).

A distance between all pairs of 14,000 patients was calculated. Pairs of patients that were the closest to each other were merged together in one cluster. Next, the next two most similar patients are grouped together. The process was continued until all the patients were clustered together. Dendrogram 400 is a visualization of the patient clustering. Axis x represents positions of each of the 14,000 patients, and axis y represents the closeness of the patient clusters. As can be seen, clusters are grouped together (blue lines connecting the clusters). The hierarchical clustering algorithm (described above) is applied, causing the clusters to grow and the distance between them to get bigger (the clusters are connected higher up with respect to the y axis). At the top on the far right side of the graph a horizontal line 460 connects a cluster 462 on the right hand side and a very big cluster 466 on the left hand side of the graph. Here, using the scaled distance method allowed identification of different groups of disease profiles that are clinically meaningful.

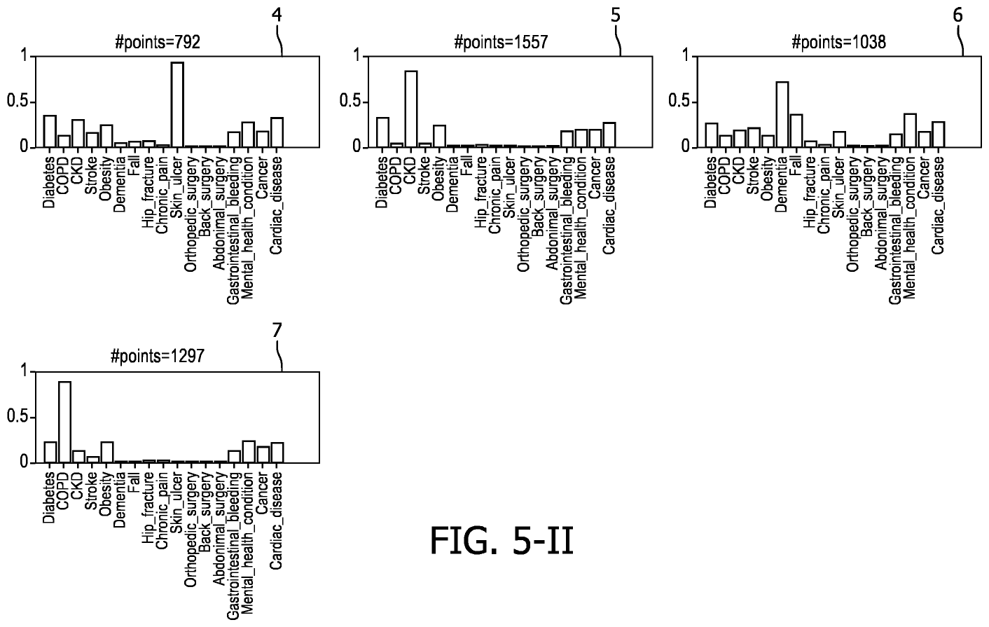

FIG. 5 illustrate the patient clusters based on disease profiles. FIG. 5 shows seven clusters representing the disease profiles of the 14000 patients in bar graphs. main group of patients with “common diseases” is grouped in cluster 2 (having a size of 7773 patients) and six satellite clusters can be identified each with having approximately 1000 patients representing similar disease profiles, yet different from the “common” group. For example, cluster 1 includes 830 patients clustered together and they have a disease profile in which all patients are susceptible to a stroke, and a limited set of other diseases like diabetes, chronic kidney disease, and cardiac disease. Cluster 3 includes 1,398 patients clustered together having a disease profile in which all patients have gastrointestinal bleeding. As can be seen from FIG. 5, each of clusters 4-7 represent a group of patients representing a similar disease profile but still different from cluster 2.

In some embodiments, the clustering algorithm based on scaled distance may be dynamically updated (e.g., as new/updated patient information is available). For example, patient information component 22 may be configured to periodically or continuously update information about the patient in the patient population (e.g., adding more patients to the population, removing patients from the population, updating medical condition status, treatment status, behavior changes, etc.). The update of the patient information triggers update of the data analysis (e.g., changes in the population, diseases, treatments, etc.) which in turn causes an update of the distance measures (including calculation of the probabilities described above), the resulting clusters, and the cluster analysis.

Electronic storage 50 includes electronic storage media that electronically stores information. The electronic storage media of electronic storage 50 may include one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 50 may be (in whole or in part) a separate component within system 10, or electronic storage 50 may be provided (in whole or in part) integrally with one or more other components of system 10 (e.g., computing devices 18, processor 20, etc.). In some embodiments, electronic storage 50 may be located in a server together with processor 20, in a server that is part of external resources 16, in a computing device 18, and/or in other locations. Electronic storage 50 may include one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 50 may store software algorithms, information determined by processor 20, information received via a computing device 18 and/or graphical user interface 40 and/or other external computing systems, information received from external resources 16, information received from sensors 14, and/or other information that enables system 10 to function as described herein.

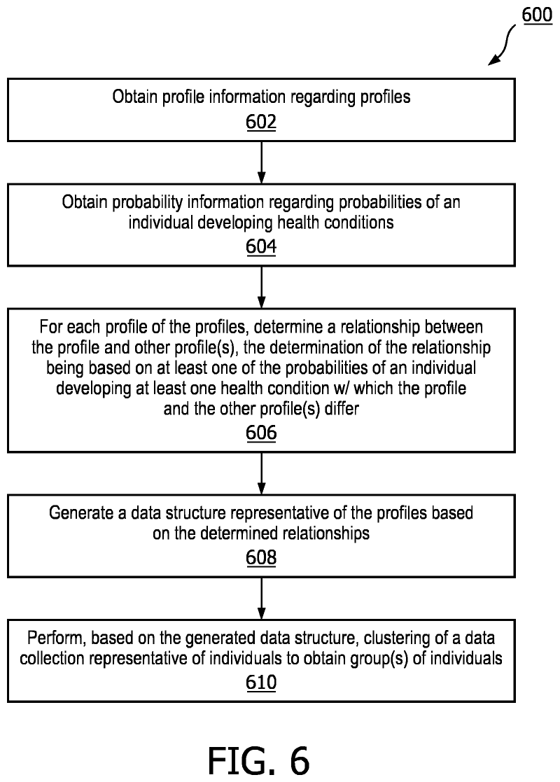

FIG. 6 illustrates a method 600 for facilitating data analysis performance with respect to analysis of individuals having one or more health conditions with a system. The system includes one or more hardware processors and/or other components. The hardware processors are configured by machine readable instructions to execute computer program components. The computer program components include a patient information component, a probability component, a data analysis component, a clustering component, a presentation component, and/or other components. The operations of method 600 presented below are intended to be illustrative. In some embodiments, method 600 may be accomplished with one or more additional operations not described, and/or without one or more of the operations discussed. Additionally, the order in which the operations of method 600 are illustrated in FIG. 6 and described below is not intended to be limiting.

In some embodiments, method 600 may be implemented in one or more processing devices (e.g., a digital processor, an analog processor, a digital circuit designed to process information, an analog circuit designed to process information, a state machine, and/or other mechanisms for electronically processing information). The processing devices may include one or more devices executing some or all of the operations of method 600 in response to instructions stored electronically on an electronic storage medium. The processing devices may include one or more devices configured through hardware, firmware, and/or software to be specifically designed for execution of one or more of the operations of method 600.

At an operation 602, profile information regarding profiles is obtained. In some embodiments, each of the profiles indicates one or more health conditions or an individual having one or more health conditions. In some embodiments, operation 602 is performed by a processor component the same as or similar to patient information component 22 and/or other components of system 10 (shown in FIG. 1 and described herein).

At an operation 604, probability information regarding probabilities of an individual developing health conditions is obtained. In some embodiments, each of the probabilities is a probability of an individual developing a health condition. In some embodiments, operation 604 is performed by a processor component the same as or similar to probability component 23 and/or other components of system 10 (shown in FIG. 1 and described herein).

At an operation 606, for each profile of the profiles, a relationship between the profile and one or more other profiles that are different from the profile is determined. In some embodiments, the relationship is determined with respect to at least one health condition, the determination of the relationship being based on at least one of the probabilities of an individual developing the at least one health condition. In some embodiments, the determination of the relationship is further based on severity related to the at least one health condition. In some embodiments, the determination of the relationship is further based on one or more costs related to the at least health condition. In some embodiments, operation 606 is performed by a processor component the same as or similar to data analysis component 24 and/or other components of system 10 (shown in FIG. 1 and described herein).

At an operation 608, a data structure representative of the profiles based on the determined relationships is generated. In some embodiments, operation 608 is performed by a processor component the same as or similar to data analysis component 24 and/or other components of system 10 (shown in FIG. 1 and described herein).

At an operation 610, clustering of a data collection representative of individuals is performed based on the generated data structure, to obtain one or more groups of individuals. In some embodiments, operation 610 is performed by a processor component the same as or similar to clustering component 26 and/or other components of system 10 (shown in FIG. 1 and described herein).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A system for facilitating clustering performance with respect to analysis of individuals having one or more health conditions, the system comprising one or more hardware processors configured by machine readable instructions to:
obtain profile information regarding at least 1000 profiles, each of the 1000 profiles indicating one or more health conditions or an individual having one or more health conditions;
obtain probability information regarding probabilities of an individual developing health conditions, each of the probabilities being a probability of an individual developing a health condition;
for each profile of the 1000 profiles, assign a distance between the profile and one or more other profiles that are different from the profile with respect to at least one health condition, the assignment of the distance being based on at least one of the probabilities of an individual developing the at least one health condition;
generate a data structure representative of the 1000 profiles with respect to a multi-dimensional binary space based on the assigned distances; and
perform, based on the generated data structure, clustering of a data collection representative of at least 1000 individuals to obtain one or more groups of individuals.

2. The system of claim 1, wherein the one or more hardware processors are configured to:
obtain patient health information regarding a patient population, the patient health information indicating health conditions of individuals in the patient population; and
obtain the probability information by determining, based on the patient health information, the probabilities of an individual developing health conditions.

3. The system of claim 1, wherein the one or more processors are further configured to:
obtain the probability information by determining a first probability of an individual having a first set of health conditions developing a second health condition not included in the first set of health conditions, wherein the probabilities comprise the first probability, the first set of health conditions comprise a first health condition;
for a first profile of the 1,000 profiles that corresponds to the first set of health conditions, assign, based on the first probability, a first distance between the first profile and a second profile that corresponds to a second set of health conditions, wherein the second set of health conditions comprises the first health condition and the second health condition; and
generate the data structure based on the first distance and one or more other distances of the assigned distances.

4. The system of claim 1, wherein the one or more processors are further configured to: generate the data structure representative of the 1000 profiles by (i) obtaining the data structure and (ii) modifying, based on the assigned distances, relationships among the 1000 profiles to reflect the assigned distances.

5. The system of claim 1, wherein the data structure comprises a graph-based data structure or a vector-based data structure, and the data structure comprises edges that reflect the assigned distances.

6. The system of claim 1, wherein the assignment of the distance is further based on severity related to the at least one health condition.

7. The system of claim 1, wherein the assignment of the distance is further based on one or more costs related to the at least health condition.

8. A method for facilitating data analysis performance with respect to analysis of individuals having one or more health conditions with a system, the system comprising one or more hardware processors configured by machine readable instructions, the method comprising:
obtaining profile information regarding profiles, each of the profiles indicating one or more health conditions or an individual having one or more health conditions;
obtaining probability information regarding probabilities of an individual developing health conditions, each of the probabilities being a probability of an individual developing a health condition;
for each profile of the profiles, determining a relationship between the profile and one or more other profiles that are different from the profile with respect to at least one health condition, the determination of the relationship being based on at least one of the probabilities of an individual developing the at least one health condition; and
generating a data structure representative of the profiles based on the determined relationships.

9. The method of claim 8, further comprising: performing, based on the generated data structure, clustering of a data collection representative of individuals to obtain one or more groups of individuals.

10. The method of claim 8, wherein the one or more processors are further configured to:
obtaining the probability information by determining a first probability of an individual having a first set of health conditions developing a second health condition not included in the first set of health conditions, wherein the probabilities comprises the first probability, the first set of health conditions comprise a first health condition;
for a first profile of the profiles that corresponds to the first set of health conditions, assigning, based on the first probability, a first distance between the first profile and a second profile that corresponds to a second set of health conditions, wherein the second set of health conditions comprises the first health condition and the second health condition; and
generating the data structure based on the first distance and one or more other distances of the assigned distances.

11. The method of claim 8, wherein the one or more processors are further configured to: generate the data structure representative of the 1000 profiles by (i) obtaining the data structure and (i) modifying, based on the assigned distances, relationships among the 1000 profiles to reflect the assigned distances.

12. The method of claim 8, wherein the data structure comprises a graph-based data structure or a vector-based data structure, and the data structure comprises edges that reflect the assigned distances.

13. The method of claim 8, wherein the determination of the relationship is further based on severity related to the at least one health condition.

14. The method of claim 8, wherein the determination of the relationship is further based on one or more costs related to the at least health condition.

15. A system for facilitating data analysis performance with respect to analysis of individuals having one or more health conditions, the system comprising:
means for obtaining profile information regarding profiles, each of the profiles indicating one or more health conditions or an individual having one or more health conditions;
means for obtaining probability information regarding probabilities of an individual developing health conditions, each of the probabilities being a probability of an individual developing a health condition;
more other profiles that are different from the profile with respect to at least one health condition, the determination of the relationship being based on at least one of the probabilities of an individual developing the at least one health condition; and;
means for generating a data structure representative of the profiles based on the determined relationships.

16. The system of claim 15, further comprising: means for performing, based on the generated data structure, clustering of a data collection representative of individuals to obtain one or more groups of individuals.

17. The system of claim 15, further comprising:
means for obtaining the probability information by determining a first probability of an individual having a first set of health conditions developing a second health condition not included in the first set of health conditions, wherein the probabilities comprises the first probability, the first set of health conditions comprise a first health condition;
means for assigning, for a first profile of the profiles that corresponds to the first set of health conditions, a first distance between the first profile and a second profile that corresponds to a second set of health conditions, wherein the second set of health conditions comprises the first health condition and the second health condition; and
means for generating the data structure based on the first distance and one or more other distances of the assigned distances.

18. The system of claim 15, further comprising: means for generating the data structure representative of the 1000 profiles by (i) obtaining the data structure and (i) modifying, based on the assigned distances, relationships among the 1000 profiles to reflect the assigned distances.

19. The system of claim 15, wherein the data structure comprises a graph-based data structure or a vector-based data structure, and the data structure comprises edges that reflect the assigned distances.

20. The system of claim 15, wherein the determination of the relationship is further based on severity related to the at least one health condition.

21. The system of claim 15, wherein the determination of the relationship is further based on one or more costs related to the at least health condition.
